(19) United States
(12) Patent Application Publication
(10) Pub. No.: US 2023/0165798 A1
(43) Pub. Date: Jun. 1, 2023

(54) MACROCYCLIC LACTONE FORMULATIONS, METHODS OF THEIR PREPARATION AND USE OF THE FORMULATIONS IN TREATING PATHOLOGIES SECONDARY TO OPHTHALMIC PARASITES

(71) Applicant: Hovione Scientia, Co. Cork (IE)

(72) Inventors: Carla Vozone, East Brunswick, NJ (US); Andreia Filipa dos Santos Cordeiro Robert Lopes, Odivelas (PT); Hugo Almeida, Coimbra (PT); Courtney Rouse Smith, Gainesville, GA (US); Sérgio Silva, Lisboa (PT); George Macgrath, Mount Pleasant, SC (US)

(73) Assignee: Hovione Scientia, Co. Cork (IE)

(21) Appl. No.: 17/284,097

(22) PCT Filed: Oct. 11, 2019
(86) PCT No.: PCT/US2019/055964

§ 371 Date: Apr. 9, 2021

(30) Foreign Application Priority Data
Oct. 12, 2018 (PT) PT115075

(51) Int. Cl. A61K 9/14; A61K 31/365; A61K 9/00; A61K 47/38; A61K 47/32

(52) U.S. Cl. CPC A61K 9/146; A61K 31/365; A61K 9/0048; A61K 47/38; A61K 47/32

(57) ABSTRACT

The invention relates to a method of treating parasitic etiologies of ophthalmic diseases in the eyelash, eyelid, or cutaneous tissue surrounding the eyelash or eyelid by topically applying to the eyelash, eyelid, or cutaneous tissue surrounding the eyelash or eyelid a formulation of antiparasitic agents such as macrocyclic lactone parasiticides, comprising of suspended particles of ivermectin and polymer solid dispersion in a suitable pharmaceutically carrier. The formulation may include particles of ivermectin and a polymer having a D90 particle size below about 10 microns preferably between about 800 nm and about 4 microns. The polymer may be an extended release polymer. The formulation may further include mineral oil and an anhydrous gel. The formulation may have a viscosity between 30,000 cP and about 100,000 cP preferably between about 40,000 cP and about 90,000 cP.

### TECHNICAL FIELD OF THE INVENTION

The invention relates to the use of macrocyclic lactone parasiticides, in particular of ivermectin and other avermectins such as doramectin and selamectin, and milbemycins such as moxidectin and milbemycin oxime, as antiparasitic agents for preparing formulations useful for treating conditions generally caused by ophthalmic parasites, in particular parasitic infections of the eye caused by Demodex mites in humans and animals. The invention also provides a method of preparing an amorphous or crystalline solid dispersion with ivermectin and a polymer. The invention also relates to the use of the formulations for treating the conditions caused by the Demodex mites in human and animals.

### BACKGROUND OF THE INVENTION

Ocular demodicosis has been identified as a pathologic overgrowth of the Demodex family of parasites, turning from a commensal relationship with the host into a parasitic relationship with the host. Demodex folliculorum and brevis are obligate parasites with a complete life cycle within and around the eyelashes, eyelash root, eyelash follicles, anterior eyelid, meibomian glands, and cutaneous periocular tissue. Infestation of the demodex in these structures may lead to meibomian gland and ocular surface inflammation, causing ocular signs and symptoms associated with inflammation of the ocular surface and eyelids (keratitis and blepharitis, respectively), and progression of the infestation may result in an evaporative dry eye disease, loss or misdirection of the eyelashes, destruction of the meibomian glands, alteration of the meibum, increased redness of the eyelids, chalazion formation, or ocular rosacea.

Between Demodex folliculorum and brevis, the folliculorum mite is the larger, measuring 0.3-0.4 mm long and typically found at the root of the eyelash. When occupying the eyelash follicle and surrounding cutaneous tissue, D. follicularis consumes and disrupts the host epithelium. This may lead to hyper keratinization, loss of the eyelash, and resultant host hypersensitivity and inflammation. Disruption of the eyelash root, eyelash follicle, and anterior eyelid including cutaneous periocular tissue may lead to signs and symptoms of eyelid and ocular surface inflammation (blepharitis and keratitis), which may lead to resultant pathologies such as evaporative dry eye disease, meibomian gland dysfunction, redness of the eyelids, chalazion formation, ocular rosacea, and loss or misdirection of the eyelashes (madarosis or trichiasis). (Luo, X., et al. (2017). “Ocular Demodicosis as a Potential Cause of Ocular Surface Inflammation.” Cornea 36 Suppl 1: S9-S14.)

Demodex brevis is smaller, measuring 0.2-0.3 mm long and typically burrows within sebaceous glands. Around the eyelid, D. brevis burrows within the meibomian gland. Infestation with D. brevis may lead to mechanical obstruction of the meibomian gland, loss of the gland architecture, or hypersensitivity and inflammation within the gland. These disruptions of the normal meibomian gland's homeostasis may lead to eyelid inflammation, chalazion formation, meibomian gland dysfunction, and ocular surface inflammation. (Luo, X., et al. (2017). “Ocular Demodicosis as a Potential Cause of Ocular Surface Inflammation.” Cornea 36 Suppl 1: S9-s14.)

While demodex is resistant to many treatments, attempts to treat demodex infestation with either topical tea tree oil or oral anti-parasitic agents have been proven effective in decreasing the signs of eyelid inflammation (Cheng, A. M., et al. (2015), “Recent advances on ocular Demodex infestation.” Curr Opin Ophthalmol 26(4): 295-300.). In a recent study of oral ivermectin, 19 subjects with confirmed infestation of demodex and concurrent ocular inflammation were treated with oral ivermectin. All subjects had eradication of the demodex by month 3 of treatment. All but two subjects improved symptomatically, and all subjects had an improvement in signs of ocular inflammation (Filho, P. A., et al. (2011). “The efficacy of oral ivermectin for the treatment of chronic blepharitis in patients tested positive for Demodex spp.” Br J Ophthalmol 95(6): 893-895.). A separate study of subjects with demodex infestations treated with topical tea tree oil proved that tea tree oil will kill demodex in a dose dependent fashion (Gao Y Y, et al. (2007) “Clinical treatment of ocular demodicosis by lid scrub with tea tree oil.” Cornea 26:136-143). While the mechanism of action is not fully known, it is hypothesized that tea tree oil cleans the epidermal debris at the eyelash root, stimulates the demodex to come to the cutaneous tissue surface, and has anti-inflammatory, anti-bacterial, and anti-fungal properties, and Terpinen-4-ol was recently identified as the molecule responsible for tea tree oil effects (Tighe, S., et al. (2013) “Terpinen-4-ol is the Most Active Ingredient of Tea Tree Oil to Kill Demodex Mites.” Transl Vis Sci Technol 2(7): 2.). Despite the efficacy of tea tree oil in eradication of the parasite, there remains no FDA approved treatment for ocular demodicosis.

One aspect of the current invention is a topical formulation of ivermectin delivered to the anterior eyelid, eyelashes, eyelash root, eyelash follicle, cutaneous periocular tissue, and meibomian gland. The formulation may be applied with fingertips or via an applicator. The applicator will both allow precision application to the site of action and simultaneous cleansing of the eyelashes and eyelash root.

Ivermectin is a commonly used anti-parasitic for demodex, although demodex is considered relatively resistant to various anti-parasitics and requires a relatively high dose to achieve sufficient eradication, particularly in the veterinary literature. While oral ivermectin is capable of eradication of eyelid demodex, a topical formulation of ivermectin or similar avermectins would allow several advantages. First, oral ivermectin has numerous side effects, including but not limited to: fever, itching, headache, skin rash, elevated liver enzymes, worsening bronchial asthma, and tachycardia and electrocardiography changes. The oral dose is contraindicated in patients with liver or kidney disease, pregnant or breastfeeding women, and children. Ivermectin has many drug-drug interactions; including but not limited to coumadin and other coumarins and vitamin K, as ivermectin is known to prolong prothrombin time. Second, ivermectin should be avoided with drugs that modulate ligand-gated chloride channels, including gated by gamma-aminobutyric acid (GABA), e.g., benzodiazepines, since the anti-parasitic mechanism occurs via nerve and muscle cells hyperpolarization through chloride ions permeation. Drugs that interact with CYP3A4 may change the metabolism of ivermectin and result in toxicity with other medications metabolized by CYP3A4 with low therapeutic indices. Third, according to its label, oral ivermectin should be taken on an empty stomach, one hour prior to eating breakfast or no food should be taken 2 hours before or after administration. These food constraints cause restrictions in the daily life of active patients (https://www.ncbi.nlm.nih.gov/pubmedhealth/PMHT0001011/; Homeida M. A. M., et al. (1988). “Prolongation of Prothrombin time with Ivermectin.” The Lancet 331(8598): 1346-1347.; Canga, A. G., et al. “The Pharmacokinetics and Interactions of Ivermectin in Humans—A Mini-review” AAPS J 10(1): 42-46 (2008); Gilbert, B. W., et al. “A Case of Ivermectin—Induced Warfarin Toxicity: First Published Report.” Hospital Pharmacy 001857871875897).

The ability to deliver ivermectin and other anti-parasitics to the anterior eyelid, eyelashes, and meibomian gland minimizes the systemic exposure to the medicine and therefore potentially decreases the risks of drug side effects, toxicities, and drug-drug interactions. Further, delivery directly to the habitat site of the demodex such as anterior eyelids/eyelashes and meibomian glands, allows high local concentrations of the anti-parasitic which may decrease the risk of developing local resistance. Moreover, local administration of the ivermectin with an applicator will have synergistic effects of both cleaning keratin debris while simultaneously eradicating the infestation. Finally, in addition to anti-parasitic effects, ivermectin has been shown to possess anti-inflammatory action by causing a decrease in TNF-α, IL-1, and IL-6 (lipopolysaccharide (LPS)-induced cytokines) through nuclear factor kappa B (NF-κB), improving the inflammatory counterpart of the disease (Zhang, X. et al. (2008) “Ivermectin inhibits LPS-induced production of inflammatory cytokines and improves LPS-induced survival in mice” Inflamm Res 57(11):524-9).

U.S. Pat. No. 9,457,038B2 (and references therein) describes prior art in the field and has similar conclusions herein that topical ivermectin would meet an unmet medical need in the treatment of ocular demodex infestations with the potential to significantly improve treatment of ocular pathologies. Of note, U.S. Pat. No. 9,457,038B2 and the referenced patent family propose delivery of ivermectin directly to the surface of the eye and only references administering directly to the conjunctiva and cornea, not tissue adjacent to the conjunctiva or cornea. Delivery to the eye deposits ivermectin near the site of the demodex infestation, but the application unnecessarily exposes the eye to high levels of ivermectin and does not deliver the anti-parasitic directly to the site of infestation. In one aspect, the current invention proposes to apply ivermectin or other macrocyclic lactose parasiticides directly to the anterior eyelids, eyelashes, eyelash root, cutaneous periocular tissue, and meibomian glands preferably via a precision applicator to target the dose of ivermectin to the sites the demodex inhabits and minimize ivermectin exposure to the eye and remainder of the body, using a sterile/aseptic semi-solid topical formulation of ivermectin.

By preferably applying ivermectin directly to the site of demodex with a precision applicator, the current invention maximizes the dose of ivermectin to the site of action and minimizes both systemic and eye exposure to ivermectin. In one aspect of the invention, the ivermectin formulation comprises a solid amorphous dispersion of ivermectin to efficiently target Demodex while decreasing ocular exposure. This formulation, combined with a particle size distribution below 4 μm, allows increased penetration of the eye lash root where the Demodex lives and prevents any mechanical irritation in the eye. Eradication of Demodex in the natural site of infestation improves the ability to eradicate ocular demodicosis and improve the symptoms of patient's suffering from this condition.

Ivermectin amorphous solid dispersions are disclosed in (a) Ivermectin-loaded microparticles for parenteral sustained release: in vitro characterization and effect of some formulation variables (J Microencapsul. 2010; 27(7):609-17) 3; (b) Sustained release ivermectin-loaded solid lipid dispersion for subcutaneous delivery: in vitro and in vivo evaluation (Drug Deliv., 2017; 24(1): 622-631); and (c) WO2016016665A1 where ivermectin amorphous solid dispersions are prepared by co-precipitation in a microfluidizator/microreactor with a stabilizing agent.

### SUMMARY OF THE INVENTION

In one general aspect, the invention relates to a method of treating ophthalmic pathologies secondary to parasitic infestations in the eyelash, eyelid, or cutaneous tissue surrounding the eyelash or eyelid by topically applying to the eyelash, eyelid, or cutaneous tissue surrounding the eyelash or eyelid a formulation comprising a solution, semi-solid, suspension or gel comprising particles of solid dispersions of ivermectin and polymer.

Embodiments of the method may include one or more of the following features. For example, the formulation may include particles of ivermectin and a polymer having a D90 particle size below 10 microns, preferably between about 800 nm and about 4 microns. The polymer may include an extended release polymer, an immediate release polymer or a mixture thereof. The polymer may be a natural or synthetic biodegradable polymer.

The natural biodegradable polymers may be one or more of, polysaccharides, cyclodextrin, chitosan, alginate and derivatives, sodium hyaluronate, xanthan gum, gellan gum, starch, proteins, albumin, gelatin, fibrins and collagen.

The synthetic biodegradable polymers comprise one or more of polyesters, polyethers, poly(anhydrides), poly(urethanes), poly(alkyl cyanoacrylates) (PACA), poly(orthoesters), cellulose and derivatives, poly(N-vinylpyrrolidones) (PVP), poly(vinyl alcohols) (PVA), and poly(acrylamides).

The polyesters may include one or more of poly(glycolic acid) (PLA), poly(1-lactic acid) (PLA), and poly(lactide-co-glycolide) (PLGA), the polyether may include one or more of poly(ethylene glycol) and poly(propylene glycol), and the cellulose and derivatives may include one or more of hydroxypropylmethyl cellulose, hydroxyethyl cellulose, hydroxyethylmethyl cellulose, hydroxypropyl cellulose, hypromellose phthalate, cellulose acetate, cellulose acetate phthalate, methylcellulose, ethylcellulose, cellulose, carboxyethylcellulose, microcrystalline cellulose and silicified microcrystalline cellulose.

The particles of ivermectin and polymer may include amorphous ivermectin or crystalline ivermectin or a co-crystal comprising ivermectin.

The formulation may further include a liquid or semi-solid pharmaceutically acceptable carrier including a polymeric gelling agent and one or more pharmaceutically acceptable excipients. The carrier is selected not to dissolve the solid dispersions of ivermectin and polymer and may be one or more of mineral oil, poloxamer 407, carbomer, methylcellulose, and sodium carboxymethyl cellulose.

The formulation may have a viscosity between about 30,000 cP and about 100,000 cP preferably 30,000 to 90,000 cP.

When applying the formulation the method further includes avoiding contact of the formulation to the conjunctiva or cornea.

In another general aspect, the invention includes a solid dispersion in the form of particles consisting essentially of ivermectin and a polymer to protect the ivermectin in the particles from terminal sterilization processes such as gamma irradiation, heat sterilization or e-beam irradiation sterilization, to increase the drug's bioavailability and to control the release of the ivermectin from the particles. The ivermectin is in an amorphous or crystalline form, the particles have a D90 particle size below 10 microns preferably of between about 800 nm and about 4 microns, and the ratio of ivermectin to polymer in the particle is about 10:1 to about 1:10 preferably 1:3 to about 4:1.

Embodiments of the solid dispersion may include one or more of the following features. For example, the polymer may be PVP VA-64 and the PVP VA-64 is present at a ratio of ivermectin to PVP VA-64 of about 1:1. The polymer may be PVP K-30 and the PVP K-30 is present at a ratio of ivermectin to PVP K-30 of about 1:3. The polymer may be HPMC-E4M and the HPMC-E4M is present at a ratio of ivermectin to HPMC-E4M of about 4:1.

The particles of the solid dispersion may include a first population of particles comprising a first ratio of ivermectin to polymer in the particle and a second population of particles comprising a second ratio of ivermectin to polymer in the particle. The first ratio and the second ratio are different, whereby the first population of particles releases the ivermectin faster than the second population of particles.

The solid dispersion may have the D90 of the first population of particles being different from the D90 of the second population of particles.

The particles of the solid dispersion may include a first population of particles comprising a first polymer in the particle and a second population of particles comprising a second polymer in the particle. The first polymer and the second polymer are different, whereby the first population of particles releases the ivermectin faster than the second population of particles.

The solid dispersion may have the D90 of the first population of particles being different from the D90 of the second population of particles.

In another general aspect, the invention relates to a pharmaceutical formulation in the form of a gel, ointment, or solution comprising of a suspended solid dispersion in an oil and a polymeric hydrocarbon gelling agent, wherein the formulation has a viscosity between about 30,000 cP and about 100,000 cP preferably 40,000 to 90,000 cP.

Embodiments of the formulation may include one or more of the following features. For example, the carrier may be one or more of polymeric hydrocarbon gels, poloxamer 407, carbomer, methylcellulose, and sodium carboxymethyl cellulose. The polymeric hydrocarbon gels can be of any suitable gelling agent and is preferably any of a gel comprising of an oil and gelling polymers.

The pharmaceutical formulation may be configured to release the ivermectin over a period of twelve hours according to standard dissolution testing methods.

The pharmaceutical formulation may be part of a kit comprising the same and a precision applicator.

The invention also relates to a method of killing demodex mites by topically applying the pharmaceutical formulation described herein to the cutaneous tissue surrounding the eyelash, eyelid and/or to the eyelash or eyelid. When applying the pharmaceutical formulation the method further includes avoiding contact of the formulation to the conjunctiva or cornea.

### DESCRIPTION OF THE DRAWINGS

FIG. 1 is a scanning electron microscopic picture of the amorphous solid dispersion (ASD) of Example 1 (ivermectin and PVP-VA-64).

FIG. 2 is a scanning electron microscopic picture of the amorphous solid dispersion of Example 2 (ivermectin and PVP K-30).

FIG. 3 is a scanning electron microscopic picture of the amorphous solid dispersion of Example 3 (ivermectin and HPMC E4M).

FIG. 4 is a thermogram of the ASD of Example 1 obtained by differential scanning calorimetry.

FIG. 5 is a thermogram of the ASD of Example 2 obtained by differential scanning calorimetry.

FIG. 6 is a thermogram of the ASD of Example 3 obtained by differential scanning calorimetry.

FIG. 7 is a diffractogram of the ASD of Example 1.

FIG. 8 is a diffractogram of the ASD of Example 2.

FIG. 9 is a diffractogram of the ASD of Example 3.

FIG. 10 is a Raman spectrum for ivermectin, the polymer PVP VA-64 and mixtures of the two.

FIG. 11 is a Raman spectrum for ivermectin, the polymer PVP K-30 and mixtures of the two.

FIG. 12 is a XRPD diffractogram of the ASD of ivermectin and PVP-VA-64.

FIG. 13 is a XRPD diffractogram of the ASD of ivermectin and PVP K-30.

FIG. 14 is a XRPD diffractogram of the ASD of ivermectin and HPMC E4M.

FIG. 15 are photographs showing the results of solubility trials on artificial sebum.

### DESCRIPTION OF THE INVENTION

In one aspect, the invention relates to a solid dispersion of an avermectin, such as ivermectin, and/or a milbemycin, and a polymer. The ivermectin may be amorphous or crystalline. For example, the amorphous solid dispersion, or ASD, may comprise amorphous ivermectin (structure provided below) or a milbemycin (structure provided below) and a pharmaceutically acceptable polymer and the dispersion used in a formulation intended for ocular drug delivery. The amorphous solid dispersion comprises ivermectin as an active ingredient and a synthetic or natural biodegradable polymer.

Ivermectin is a mixture in the ratio of approximately 80:20 of 22,23-dihydro C-076 B1a and B1b.

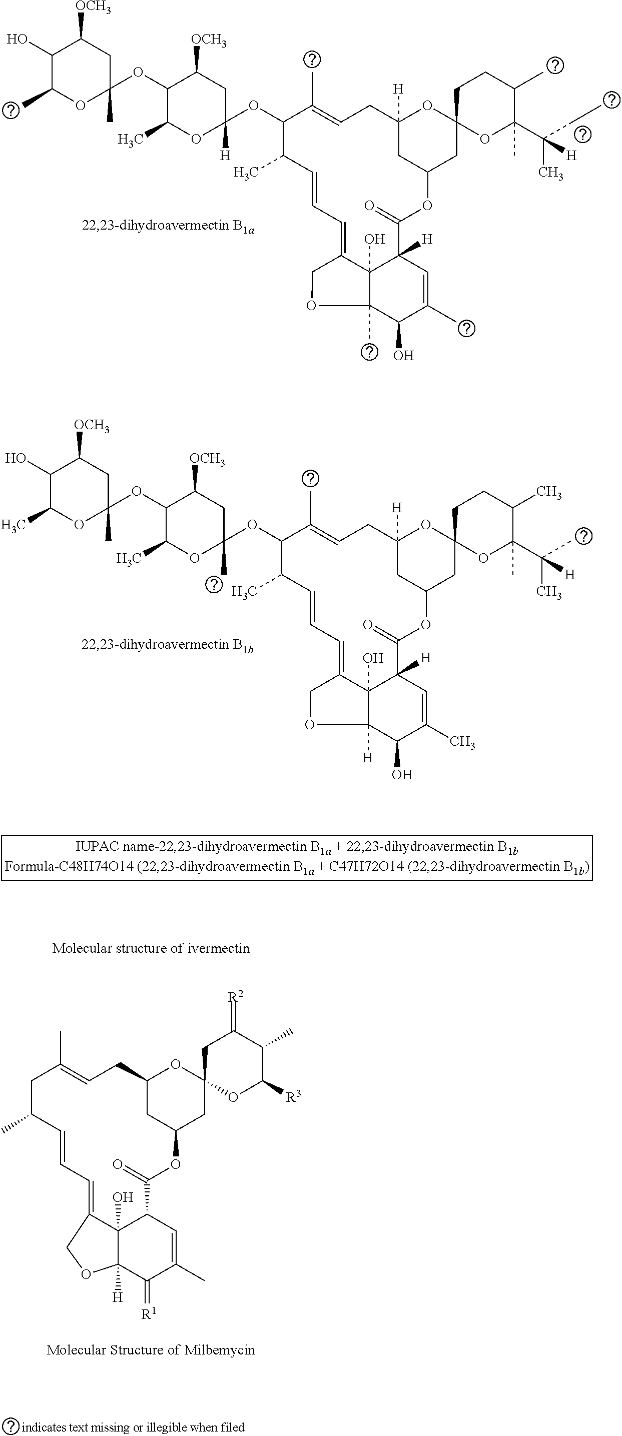

The present invention includes a method for the production of an amorphous solid dispersion (ASD) with ivermectin and a polymer that can be formed with different ratios of ivermectin:polymer. The process comprises an isolation step of spray drying a solution of ivermectin and at least one polymer in a solvent. Preferably, the solvent is an organic solvent or mixture of organic solvents, or water or mixtures thereof, such as ethanol or methanol. The production of the ASD consists first in dissolving the ivermectin in the solvent and then the addition of the polymer to the solution until complete dissolution is achieved. The solvent is removed by a solvent evaporation method such as spray drying Gas anti-solvent technique, Solvent evaporation, Solvent method, Hot Melt Extrusion, Electrospinning method, Rotary method, Fluid Bed drug layering, Fusion method, Cryogenic grinding method, Mechanical activation method, Freeze drying, Supercritical fluid, Film freezing and Agitation granulation method preferably by feeding the solution to a spray dryer and collecting the particles of the solid dispersion. The ASD can be stored at room temperature and remains stable after at least two months of storage.

More specifically, the method of preparing the ASD includes incorporating the ivermectin particles into a polymer matrix by spray drying a solution of ivermectin and a polymer in a solvent. A range of ivermectin concentrations can be used to prepare an amorphous solid dispersion. For example, in one aspect, a concentration of ivermectin between 0.01% and 30% (W/W) in the solution is preferred, more preferably between 0.1% and 30% or 0.5% and 10% and most preferably between 1% and 5%.

The polymer used in the ASD may be a natural or synthetic biodegradable polymer. The natural biodegradable polymers used include, but are not limited to, polysaccharides such as cyclodextrin, chitosan, alginate and derivatives, sodium hyaluronate, xanthan gum, gellan gum and starch, and proteins such as albumin, gelatin, fibrins and collagen.

The synthetic biodegradable polymers used include, but are not limited to, polyesters such as poly(glycolic acid) (PGA), poly(1-lactic acid) (PLA), poly(lactide-co-glycolid acid) (PLGA); polyether such as poly(ethylene glycol), poly(propylene glycol); poly(caprolactones) (PCL); poly(anhydrides); poly(urethanes); poly(alkyl cyanoacrylates) (PACA); poly(orthoesters); cellulose and derivatives such as hydroxypropyl methyl cellulose, hydroxyethyl cellulose, hydroxyethyl methyl cellulose, hydroxypropyl cellulose, hypromellose phthalate, cellulose acetate, cellulose acetate phthalate, methylcellulose, ethyl cellulose, cellulose, carboxymethyl cellulose, microcrystalline cellulose and silicified microcrystalline cellulose; poly(N-vinylpyrrolidones) (PVP); poly(vinyl alcohols) (PVA) and poly(acrylamides).

The structures of two such polymers, poly(vinylpyrrolidone (PVP) and hydroxypropyl methyl cellulose (HPMC) are provided below:

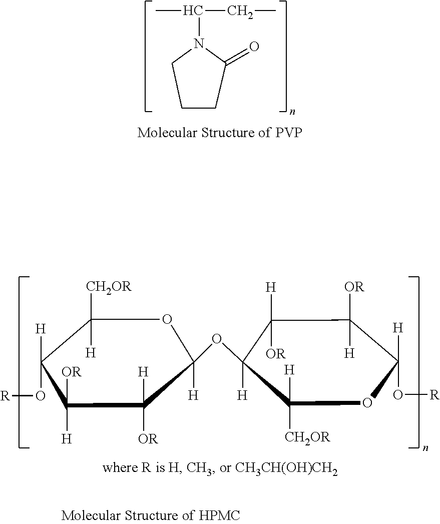

The solvent used can be an organic solvent or mixtures of organic solvents, or water or mixtures thereof. The method of preparing the amorphous ivermectin solid dispersion consists of using a suitable spray dryer, such as a lab scale spray dryer. More specifically, the method for preparing the amorphous ivermectin solid dispersion by the spray drying technique of the present invention includes the following steps:

1. Preparing the spray solution containing ivermectin and the polymer in a solvent.

2. Forming the solid dispersion by spraying the solution of Step 1) via a nozzle to obtain a solid dispersion.

3. Collecting the solid dispersion prepared in Step 2).

The amorphous solid dispersion (ASD) can be obtained by any suitable or commercially available spray dryer. The parameters of the equipment can be adjusted to obtain the ASD, namely pneumatic spray nozzle orifice, atomization gas flow, solution flow rate, drying temperature and outlet temperature.

The pneumatic spray nozzle orifice can be for example 0.7 mm and with alternative atomization methods there may be used a rotary, pressure or ultrasonic nozzle.

Any suitable drying temperature can be used, and the outlet temperature range may be from 20° C. to 100° C., preferably 30° C. to 50° C. and more preferably 40° C. to 45° C.

The drying gas flow rate for a small-scale spray dryer may be from about 20 kg/h to about 120 kg/h, preferably from about 40 kg/h to about 80 kg/h, most preferably about 40 kg/h.

The preferential atomization gas flow can be 150 to 300 milliliters per hour and can be adjusted to the equipment in use.

This method allows in just one-step a process that incorporates the ivermectin into the polymer and reaches the particle size suitable for an ophthalmic formulation. Further, this method produces a stable solid amorphous ivermectin dispersion with a particle size in the micrometer and sub-micrometer range, more specifically with a d90 that is less than 10 micrometers, preferably less than 4 micrometers. By particle size in the micrometer and sub-micrometer range, it is meant that the particles of the solid dispersion of polymer and ivermectin are of a micrometer to sub-micrometer range. It should be understood that the solid dispersion refers to a dispersion of ivermectin particles in a solid matrix of the polymer.

The ASD formed as described above is incorporated in a suspension of a vehicle in the form of a gel, to formulate an ophthalmic ointment. The resulting ASD formulation is a suitable drug delivery system and will allow a controlled release of the ivermectin, increased bioavailability of the ivermectin, and stability of the ivermectin at the site of action.

The inventor has determined that because an ophthalmic formulation must be sterilized, the Gamma irradiation, e-beam sterilization or heat sterilization methods are the most appropriate method because is not possible to sterilize a suspension by filtration. Advantageously, the polymer in the ASD protects the ivermectin from degradation during the irradiation process. The thus sterilized formulation (ASD, vehicle and other excipients as needed) may be advantageously applied with an applicator to the eyelid and at the base of eyelashes. In one embodiment, when applying the formulation the user may avoid contacting the formulation with the conjunctiva or cornea. By such specific topical application of the formulation, a patient can be treated for ocular conditions caused by infestations of Demodex.

Method of forming Amorphous Solid Dispersion of Ivermectin and Polymer.

Table 1 below provides three examples of the composition of amorphous solid dispersions of ivermectin with different polymers. Example 1 is an ASD of ivermectin with the PVP VA-64, Example 2 is an ASD of ivermectin with PVP K-30 and Example 3 is an ASD of ivermectin with HPMC E4M.

The first step in forming the ASD is preparation of a feed solution for the spray drying apparatus. Initially, the ivermectin is dissolved in the solvent. In Examples 1 and 2 ivermectin was dissolved in absolute ethanol and in Example 3 ivermectin was dissolved in a mixture of ethanol and water. In this step, the ivermectin was dissolved in a mass proportion 1% (W/V) in absolute ethanol for Example 1, 2% (W/V) in absolute ethanol for Example 2, and 0.88% (W/V) in a mixture of ethanol/water in Example 3.

With the ivermectin dissolved in the respective solvent, the polymer was next dissolved in the solution of ivermectin and solvent. In Example 1, the PVP VA-64 was added at a ratio of ivermectin to PVP VA-64 of 1:1. In Example 2, the PVP K-30 was added at a ratio of ivermectin to PVP K-30 of 1:3. In Example 3, the HPMC-E4M was added at a ratio of ivermectin to HPMC-E4M of 4:1. In this step, after complete dissolution of the ivermectin in the solvent, the polymer was added in a mass proportion of 1% (W/V) for Example 1, 6% (W/V) for Example 2, and 0.22% (W/V) for Example 3 until a clear solution was formed. Dissolution of the ivermectin and polymer was performed at room temperature.

TABLE 1
Summary of the Feed Solution Composition

| Solution parameters | Example 1 | Example 2 | Example 3 |
|---|---|---|---|
| Ivermectin (g) | 1 | 1 | 2 |
| PVP VA-64 (g) | 1 | — | — |
| PVP K-30 (g) | — | 3 | — |
| HPMC E4M (g) | — | — | 0.5 |
| Absolute ethanol (ml) | 100 | 50 | 150 |
| Water (ml) | — | — | 75 |

The next step in production of the amorphous solid dispersion is spray drying of the feed solution. In the formulations of Examples 1-3, a lab BUCHI™ B-290 Mini Spray Dryer was used to prepare the amorphous solid dispersion. The spray dryer was equipped with a two-fluid nozzle and was operated in an open cycle mode. The solutions prepared above were fed to the nozzle by a peristaltic pump and atomized at the tip of the nozzle. The particles produced were dried by a co-current of nitrogen and were collected at the bottom of the cyclone. Table 2 reports the spray dryer parameters used for each of the three formulations.

TABLE 2
Summary of the main operating conditions for Examples 1, 2 and 3

| Spray Dryer parameters | Example 1 | Example 2 | Example 3 |
|---|---|---|---|
| T_in (° C.) | 55 | 55 | 63 |
| T_out (° C.) | 40 | 40 | 40 |
| F_drying (N₂) (kg/h) | 40 | 40 | 40 |
| Rotameter level (Mm) | 40 | 40 | 52 |
| T_Feed (° C.) | RT | RT | RT |
| F_Feed (ml/min) | 2.5 | 2.5 | 2.5 |
| Nozzle (mm) | 140 | 140 | 140 |

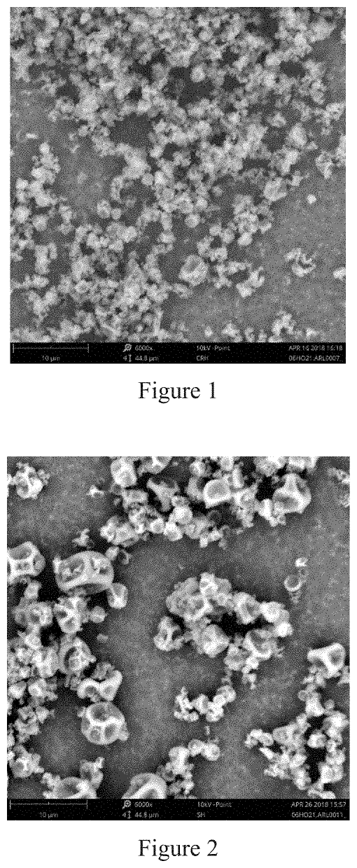

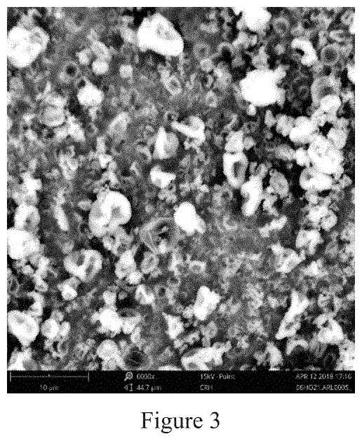

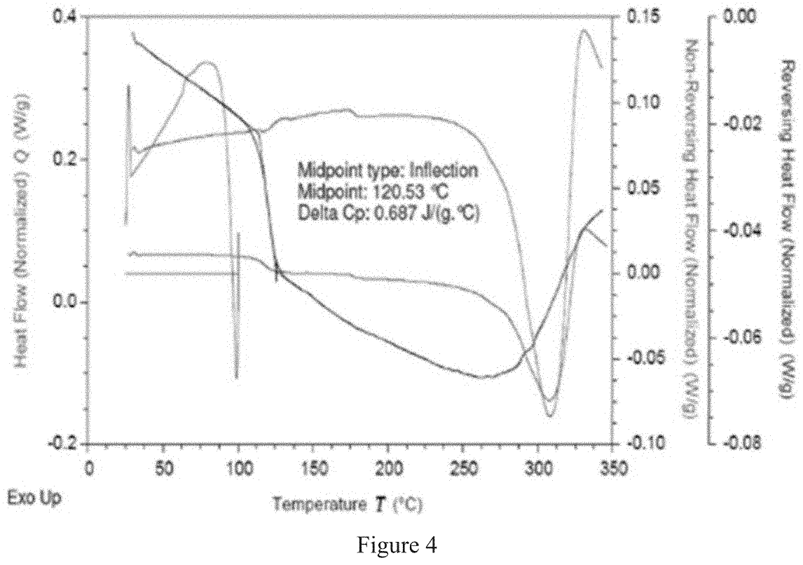

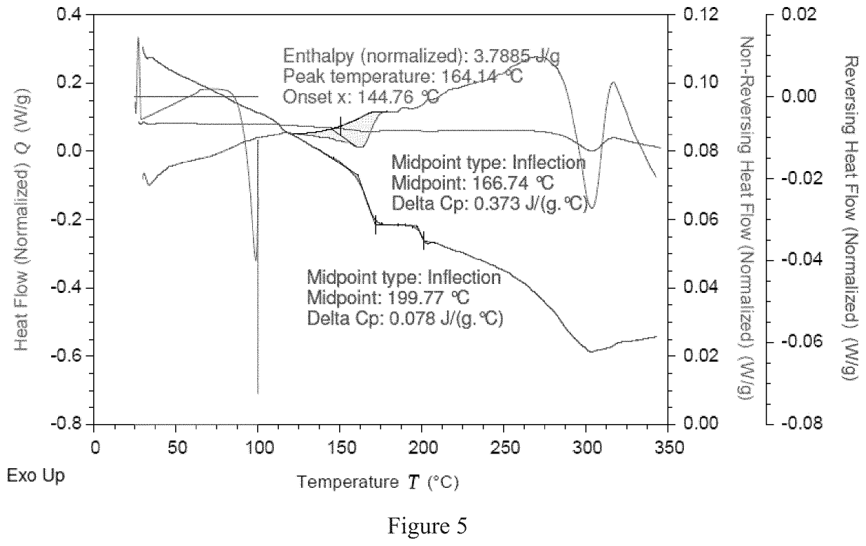

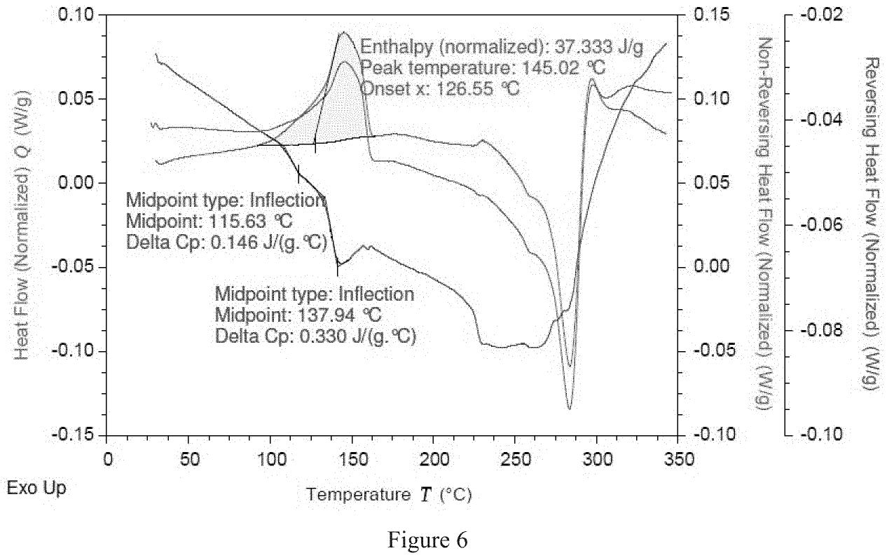

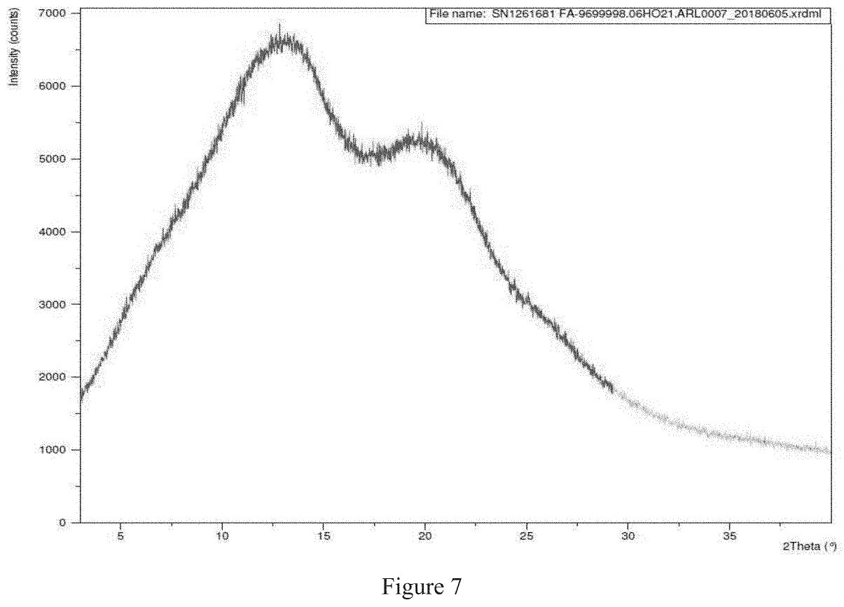

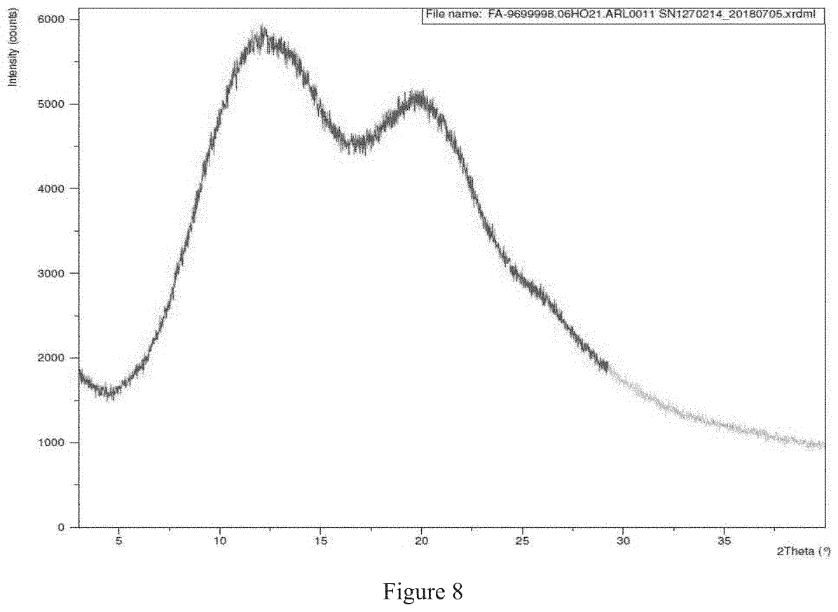

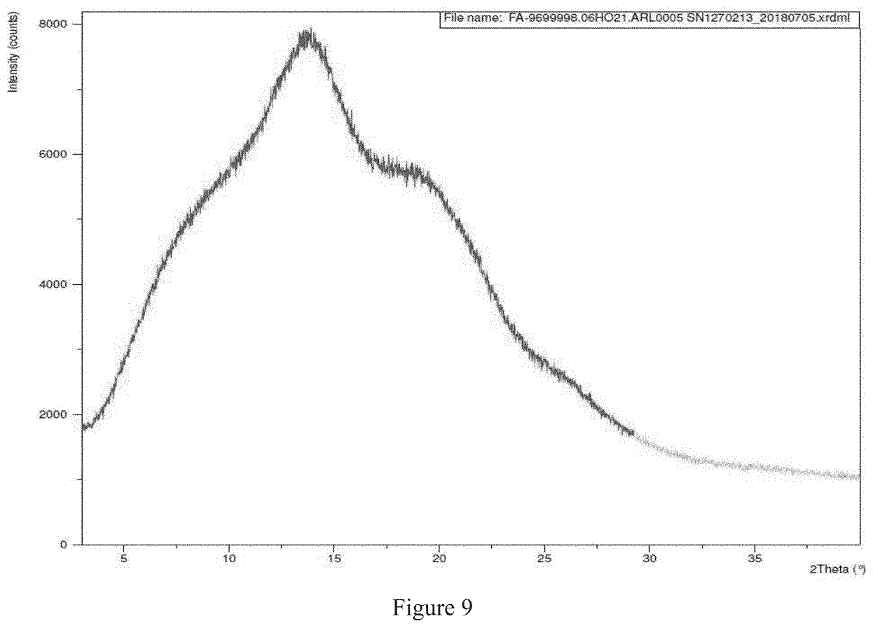

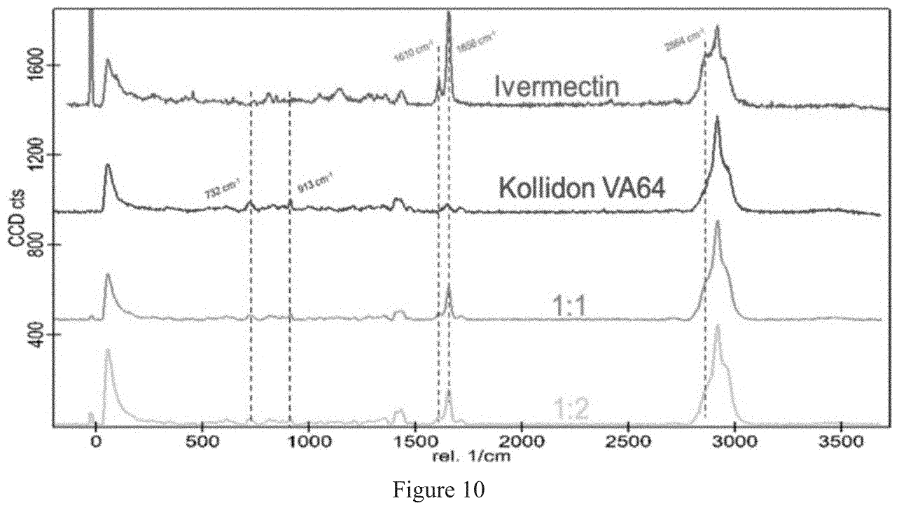

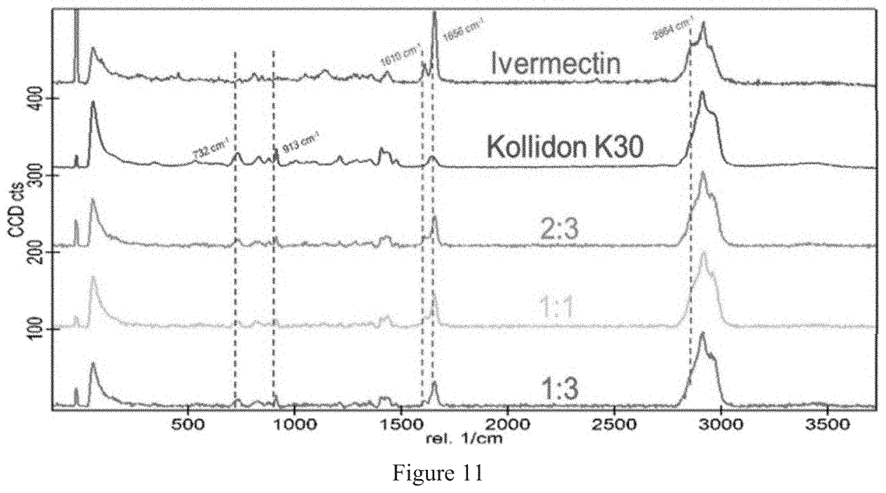

The solid-state characterization of the spray dried amorphous ivermectin solid dispersions of Examples 1-3 prepared by the traditional spray drying process were evaluated by Scanning Electronic Microscopy (SEM) (Phenom ProX SEM), Differential Scanning calorimetry (DSC) (TA Instruments), Laser diffraction (Sympatec HELOS/RODOS, Germany) employing the rotary feeder and R1 lens, X-Ray Powder Diffraction (Pan Analytical), Raman Spectroscopy (Witec) and High Performance Liquid Chromatography (Waters). FIG. 1 is a scanning electron microscopic picture of the amorphous solid dispersion of Example 1 (ivermectin and PVP-VA-64). FIG. 2 is a scanning electron microscopic image of the amorphous solid dispersion of Example 2 (ivermectin and PVP K-30). FIG. 3 is a scanning electron microscopic picture of the amorphous solid dispersion of Example 3 (ivermectin and HPMC E4M). FIGS. 1-3 are at a magnification of 6000×. FIGS. 4-6 are thermograms of the ASD of Examples 1-3 obtained by differential scanning calorimetry. FIGS. 7-9 are diffractograms of the ASD of Examples 1-3. The diffractograms demonstrate that the ASDs prepared in Examples 1-3 are amorphous. FIGS. 10 and 11 are Raman spectrums for ivermectin, the polymer PVP VA-64 and mixtures of the two (FIG. 10) and ivermectin, the polymer PVP K-30 and mixtures of the two (FIG. 11). Comparing the spectrums of the polymer with the mixtures of ivermectin and polymer shows the peaks of the mixtures to be identical with the spectrum of the polymer, which indicates a good incorporation of the ivermectin into the polymer. FIG. 10 includes ratios of ivermectin to PVP VA-64 at ratios of 1:1 and 1:2 and FIG. 11 includes ratios of ivermectin to PVP K-30 at ratios of 1:1, 1:3, and 2:3.

The inventors have also determined that use of the polymer in the amorphous solid dispersion of ivermectin protects the ivermectin from degradation that occurs during Gamma irradiation, heat sterilization or e-beam sterilization. Ophthalmic formulations must be sterilized and Gamma irradiation, heat sterilization or e-beam sterilization are suitable methods for sterilization because other methods, such as filtration, are not suitable for a suspension formulation. The amorphous solid dispersions for Examples 1-3 were tested to determine the extent that the polymer protects the ivermectin during Gamma irradiation. For preliminary tests, the amorphous ivermectin solid dispersions were sterilized by Gamma irradiation at 25 kGy for 22 hours in a Precisa 22 equipment. The ASDs were analyzed by XRPD and HPLC to determine if the Gamma irradiation changed the ivermectin polymorphic form and degradation respectively.

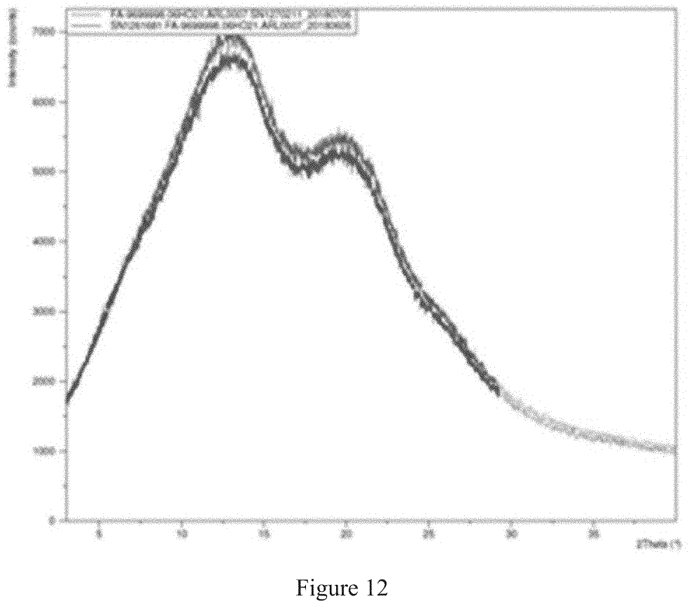

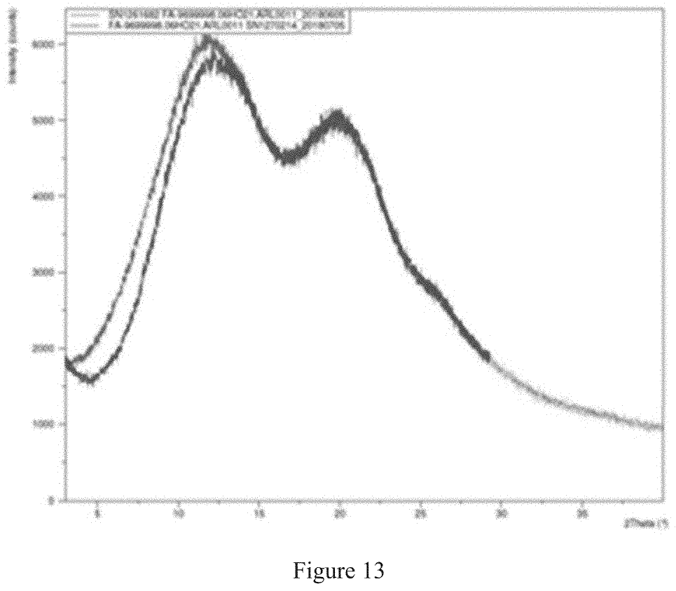

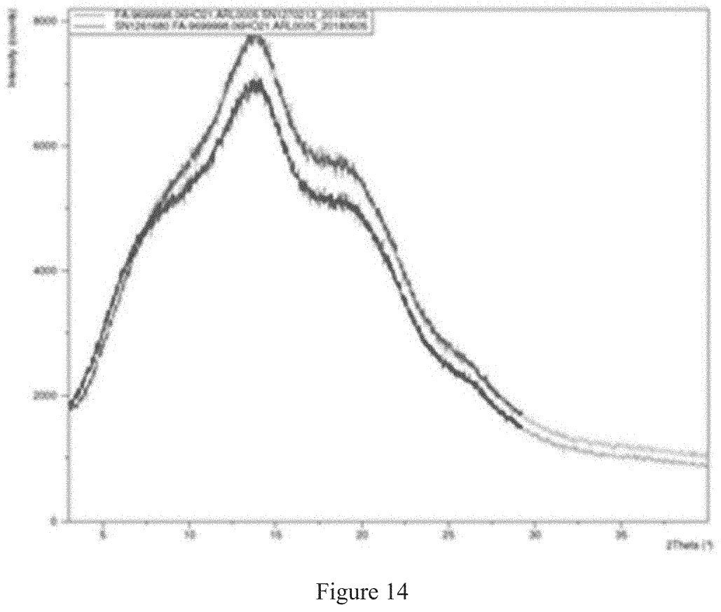

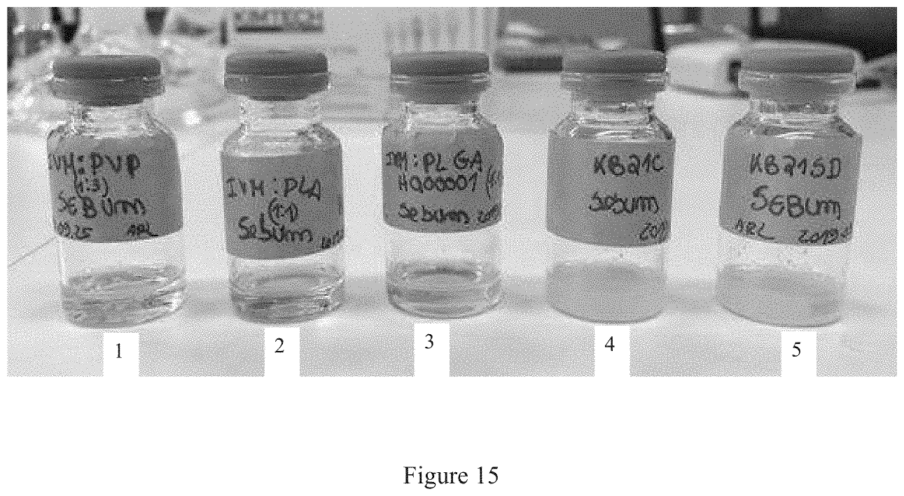

The ASDs were characterized by XRPD before and after 1-month irradiation. FIG. 12 is a diffractogram of the ASD of ivermectin and PVP-VA-64. FIG. 13 is a diffractogram of the ASD of ivermectin and PVP K-30. FIG. 14 is a diffractogram of the ASD of ivermectin and HPMC E4M. FIGS. 12-14 demonstrate that after the ASDs of Examples 1-3 remain amorphous 1 month after Gamma irradiation. This permits the ASDs to be used in a formulation and sterilized by Gamma irradiation without change in polymorphic form.

Table 3 shows the results obtained from HPLC. The assay of the ivermectin on the amorphous solid dispersion was performed before, 1 week and 1 month after the application of Gamma irradiation. Preliminary trials of sterilization by gamma irradiation were successfully completed in the solid material of ivermectin alone and ASDs. Although the amorphous form of the ASDs was not affected by gamma irradiation, Table 3 shows the protective effect of the polymer on the ivermectin during gamma irradiation. Of note, the PVP K-30 demonstrated a lower level of API degradation after gamma irradiation.

TABLE 3
Ivermectin degradation before, 1 week
and 1 month after Gamma irradiation

| | | % API | % API | |
|---|---|---|---|---|
| | % API | (After: 1 | (After: 1 | % API |
| Examples | (Before) | week) | month) | degraded |
| Example 1: IVM + | 94.4 | 89.7 | 89.3 | 5.1 |
| PVP VA-64 | | | | |
| Example 2: IVM + | 95.0 | 92.5 | 93.3 | 1.7 |
| PVP K-30 | | | | |
| Example 3: IVM + | 90.4 | 86.6 | 78.5 | 11.9 |
| HPMC E4M | | | | |
| IVM | 95.2 | 86.9 | 84.3 | 10.9 |

Table 4 provides a summary of the characterizations (HPLC, XRPD and particle size) of ivermectin and of the amorphous solid dispersion of Example 1 (PVP-VA 64), Example 2 (PVP K-30) and Example 3 (HPMC E4M) prior to irradiation and after irradiation. Preliminary trials of sterilization by gamma irradiation were successfully completed in the solid material of ivermectin alone and ASDs. The amorphous form of the ASDs was not affected by gamma irradiation. The results provided in Table 4 demonstrate a polymer protective effect of the API at different levels, with PVP K-30 showing a lower API degradation.

TABLE 4
Summary of characterization of the amorphous solid dispersion
of ivermectin and the ASDs of Example 1 (PVP-VA 64),
Example 2 (PVP K-30) and Example 3 (HPMC E4M)

| | | | After | After |
|---|---|---|---|---|
| | | | irradiation | irradiation |
| | Characterization | Before | (T = 1 | (T = 1 |
| Samples | technique | irradiation | week) | month) |
| IVM | HPLC | 95.2% | 86.9% | 84.26% |
| | (Assay IVM) | | | |
| | XRPD | Amorphous | Amorphous | Amorphous |
| | D90 (μm) | 2.66 | 2.53 | 2.41 (2 M) |
| IVM + | HPLC | 94.4% | 86.69% | 89.3% |
| Kollidon | (Assay IVM) | | | |
| VA 64 | XRPD | Amorphous | Amorphous | Amorphous |
| | D90 (μm) | 2.96 | 2.96 | 2.75 (2 M) |
| IVM + | HPLC | 95.0% | 92.5% | 93.3% |
| Kollidon | (Assay IVM) | | | |
| 30 | XRPD | Amorphous | Amorphous | Amorphous |
| | D90 (μm) | 4.97 | 5.19 | 4.68 (2 M) |
| IVM + | HPLC | 90.4% | 86.6% | 78.47% |
| Methocel | (Assay IVM) | | | |
| E4M | XRPD | Amorphous | Amorphous | Amorphous |
| | D90 (μm) | 3.75 | 5.94 | 5.44 (2 M) |

In another aspect, the invention includes a topical formulation and the ability to use a kit comprising the formulation and an applicator for use in treating ocular conditions caused by a Demodex infestation. The topical formulation kit can be used to apply the formulation with an applicator to surfaces other than the eye, including being applied to the anterior eyelid, eyelashes, eyelash root, eyelash follicle, cutaneous periocular tissue, and meibomian gland via an applicator. The applicator allows both precise application to the site of action and simultaneous cleansing of the eyelashes and eyelash root.

The inventors have determined that by applying ivermectin directly to the site of demodex with a precision applicator, this aspect of the invention maximizes the dose of ivermectin applied to the site of action and minimizes both systemic and eye exposure to ivermectin. The kit comprises the formulation and the applicator. The applicator must be sterile and can be disposable. The ivermectin formulation comprises the solid amorphous dispersion of ivermectin described above with a particle size distribution equal to or less than 10 microns. The kit and formulation are believed to efficiently target Demodex while decreasing ocular exposure. The particle size distribution of less than 10 microns allows increased penetration of the eye lash root where the Demodex lives and prevents any mechanical irritation in the eye. Eradication of demodex in the natural site of infestation improves the ability to eradicate ocular demodicosis and improve the symptoms of patient's suffering from this condition. An advantage of the invention can be the anti-inflammatory action of ivermectin in treating the condition. In this manner ivermectin can be used as an anti-inflammatory.

The topical formulation to treat Demodex infestation comprises the amorphous ivermectin solid dispersion, a carrier and other excipients. Specifically, the topical pharmaceutical formulation comprises an amorphous solid dispersion containing amorphous ivermectin and a natural or synthetic biodegradable polymer, suspended in a gel (carrier), such as Versagel®, and at least one or more of the following elements: mineral oil USP Grade, preservatives such as benzalkonium chloride, chlorobutanol, sodium perborate, stabilized oxychloro complex, chlorhexidine acetate (CHA) and phenylmercuric nitrate or acetate; antioxidants such as vitamin E and derivatives, vitamin C, beta carotene, zinc, lutein, anthocyanin's and carotenoids and sodium chloride and/or hydrochloric acid to adjust pH.

Versagel® is a commercially available mixture of gelling compositions, including Versagen M C, Versagel M D, Versagel M E, Versagel M G, Versagel M L, Versagel M N, Versagel M P, Versagel M, Versagel P, Versagel S, and Versagel S Q. The Versagel M C, MD, ME, MG, ML, MN, MP and M series include isohexadecane (MC), isododecane (MD), hydrogenated polyisobutene (ME), hydrogentated poly (C16-14 olefin) (MG), C12-15Alkyl benzoate (ML), isononyl isononanoate (MN), isopropyl palmitate (MP), mineral oil (M), petrolatum (P), hydrogenated polyisobutene (S), or squalene (SQ) with one or more of Ethylene/Propylene/Styrene Copolymer, Butylene/Ethylene/Styrene Copolymer, pentaerythrityl tetra-di-t-butyl hydroxyhydrocinnamate, dibutyl lauroyl glutamide, The Versagel® series are available in a wide range of viscosities.

In the formulation, the ivermectin range can be between 0.001% and 5% and more preferably between 0.01% and 3%. The ivermectin can be present in intermediate amounts such as 0.1%, 0.2%, 0.3%, 0.4%, 0.5%, 0.7%, 0.8%, 0.9%, 1%, 1.5%, 2%, 2.5%, 3%. The polymer range can be between 0.01% and 5% and more preferably between 0.02% and 3%. The particle size should be below 10 μm preferably between a d90<4 μm, to avoid eye irritation, and a d90>800 nm to avoid absorption inside the follicle.

The carrier used can be a gel, semi-solid, liquid or ointment. The gel may be selected from gel materials such as anhydrous gels, poloxamer 407, carbomer, methylcellulose, and sodium carboxymethyl cellulose. The viscosity of the formulation ideally should be between about 30,000 to about 100,000 cP. preferably between about 40,000 to about 90,000 cP. The objective of the viscosity is to be sufficiently thin to be applicable but sufficiently thick to remain on the tissue to which the topical formulation is applied.

Example 4 is one formulation example of a topical formulation of the amorphous solid dispersion of ivermectin in a gel. The formulation is prepared using the amorphous solid dispersion of ivermectin prepared as described above. The formulation is prepared using conventional formulation techniques.

TABLE 5
Example 4 - ASD Topical Formulation

| Ingredient | Weight Percentage |
|---|---|
| Ivermectin | 1% |
| PVP K-30 | 3% |
| Mineral oil | 5% |
| Carrier gel | 91% |
| Preservatives and antioxidants | As needed |
| Sodium Chloride and Hydrochloric Acid | To adjust pH |
| Total | 100% |

Example 4 is one formulation example of a topical formulation of the amorphous solid dispersion of ivermectin in a gel. The formulation is prepared using the amorphous solid dispersion of ivermectin prepared as described above. The formulation is prepared using conventional formulation techniques.

Variations in the above are contemplated. For example, the amorphous ivermectin solid dispersion may be in the form of a crystalline ivermectin solid dispersion.

The solid dispersion, whether amorphous or crystalline, may be formed by spray drying, or another process such as extrusion/spheronization and co-precipitation, Gas anti-solvent technique, Solvent evaporation, Solvent method, Hot Melt Extrusion, Electrospinning method, Rotary method, Fluid Bed drug layering, Fusion method, Cryogenic grinding method, Mechanical activation method, Freeze drying, Supercritical fluid, Film freezing and Agitation granulation method.

The formulation may be tested to determine its efficacy by applying to eyelashes with a demodex infestation. Prior to applying the formulation, a sample of eyelashes may be removed and analyzed by microscopy to give a baseline Demodex count. Following application of the formulation for a one week to one month, a sample of eyelashes may be removed and again analyzed by microscopy to give a Demodex count after treatment. The objective would be to reduce the levels of Demodex on the eyelashes to normal levels.

The invention also relates to a release profile of the ivermectin from the formulation. The inventors have determined that the treatment is most efficacious if the formulation releases an initial burst of ivermectin followed by a continuous release of ivermectin. A number of methods may be used to provide this dual release profile. For example, two types of ivermectin-polymer particles may be produced: a first population of particles with a relatively fast release polymer and a second population of particles with a relatively slow release polymer. The fast release polymer particles will provide the initial release of ivermectin and the slow release polymer particles will provide the continuous release of ivermectin. As a second aspect, the two types of particles can vary based on the proportion of polymer to ivermectin in each particle. Particles with a greater proportion of ivermectin will provide the initial burst and those with a reduced proportion of ivermectin will provide the continuous release of ivermectin. As a third aspect, a single population of particles can be used in which the spray drying is varied to provide an inner layer that has a greater proportion of polymer to ivermectin and an outer layer that has a greater proportion of ivermectin to polymer. The outer layer provides the initial burst of ivermectin and the inner layer provides the continuous release of ivermectin.

Some polymers have a capacity to improve the solubility of the ivermectin in the local region of application. In order to improve the solubility of the ivermectin in sebum (mites typically live in the follicles of the eyelashes where sebum is the medium, some polymers were tested, as can be seen in Example 5 (Table 7). In this example the ivermectin has to dissolve in sebum to provide the efficacy in killing the mites. An artificial sebum was formulated according to the literature, and the components are listed in Table 6.

TABLE 6
Components of the artificial sebum

| Component | Amount (g) | % |
|---|---|---|
| Squalene | 15 | 15 |
| Paraffin liq. | 10 | 10 |

Triglycerides

| | | |
|---|---|---|
| Olive oil | 10 | 10 |
| Cotton seed oil | 25 | 25 |
| Coconut oil | 10 | 10 |

Fatty acids

| | | |
|---|---|---|
| Oleic acid | 15 | 15 |
| Jojoba oil | 10 | 10 |

Cholesterol

| | | |
|---|---|---|
| Glyceryl Trioleate | 5 | 5 |
| Total | 100 (g) | 100 (%) |

The data in Table 7 shows visual solubility of three amorphous solid dispersions, with the same amount of ivermectin in each dispersion, with the polymers PVP-K30, PLA and PLGA, amorphous and crystalline ivermectin, in the artificial sebum. The sebum with the amorphous ivermectin dispersions remains clear (solubilized) after 24 h of mixing with a magnetic stirrer at room temperature, and without polymers the sebum continued opaque (ivermectin in suspension—not solubilized) after the addiction of the both forms of ivermectin (crystalline or amorphous). Thus, this shows that the polymers greatly improve the solubility of ivermectin in sebum providing the delivery of the ivermectin directly where the mites are hosted (follicles).

TABLE 7
Visual solubility of the ASD and both forms of ivermectin in artificial sebum

| | IVM:PVP-K30 | IVM:PLA | IVM:PLGA | Crystalline | Amorphous |
|---|---|---|---|---|---|
| Component | (1:3) | (1:1) | (1:1) | ivermectin | ivermectin |
| Vial | 1 | 2 | 3 | 4 | 5 |
| Solubility | clear | clear | clear | opaque | opaque |
| (visual inspection) | | | | | |

In another aspect, the formulation may comprise crystalline or amorphous ivermectin suspended in a mineral oil carrier, or in a mixture of mineral oil and gellants.

We claim:

1. A method of treating inflammation and ophthalmic pathologies secondary to parasitic infestations in the eyelash, eyelid, or cutaneous tissue surrounding the eyelash or eyelid, by topically applying to the eyelash, eyelid, or cutaneous tissue surrounding the eyelash or eyelid a formulation comprising a suspension of a solid dispersion of an avermectin and/or a milbemycin and polymer in a liquid or semi-solid carrier in which the avermectin or milbemycin is minimally soluble or not soluble.

2. The method according to claim 1, wherein the parasitic infestation comprises demodex.

3. The method according to claim 1, wherein the pathologies related to demodex infestation in the eyelash, eyelid, or cutaneous tissue surrounding the eyelash include meibomian gland dysfunction with or without evaporative dry eye disease, posterior blepharitis, anterior blepharitis, periocular dermatitis, chalazion, trichiasis or madarosis, and other conditions found to be secondary to demodex or other parasitic infestations.

4. The method of claim 1, wherein the avermectin comprises ivermectin.

5. The method of claim 4, wherein the formulation comprises particles of ivermectin and a polymer having a D90 particle size below about 10 microns preferably between about 800 nm and about 4 microns.

6. The method of claim 5, wherein the polymer comprises an extended release polymer, an immediate release polymer or a mixture thereof.

7. The method of claim 6, wherein the polymer comprises a natural or synthetic biodegradable polymer.

8. The method of claim 7, wherein the natural biodegradable polymers comprises one or more of, polysaccharides, cyclodextrin, chitosan, alginate and derivatives, sodium hyaluronate, xanthan gum, gellan gum, starch, proteins, albumin, gelatin, fibrins and collagen.

9. The method of claim 7, wherein the synthetic biodegradable polymers comprises one or more of polyesters, polyethers, poly(anhydrides), poly(urethanes), poly(alkyl cyanoacrylates) (PACA), poly(orthoesters), cellulose and derivatives, poly(N-vinylpyrrolidones) (PVP), poly(vinyl alcohols) (PVA), and poly(acrylamides).

10. The method of claim 9 wherein the polyesters comprise one or more of poly(glycolic acid) (PGA), poly(1-lactic acid) (PLA), and poly(lactide-co-glycolide) (PLGA), the polyether comprises one or more of poly(ethylene glycol) and poly(propylene glycol), and the cellulose and derivatives comprises one or more of hydroxypropyl methyl cellulose, hydroxyethyl cellulose, hydroxyethyl methyl cellulose, hydroxypropyl cellulose, hypromellose phthalate, cellulose acetate, cellulose acetate phthalate, methylcellulose, ethyl cellulose, cellulose, carboxymethylcellulose, microcrystalline cellulose and silicified microcrystalline cellulose.

11. The method of claim 5, wherein the particles of ivermectin and polymer comprise amorphous ivermectin.

12. The method of claim 5, wherein the particles of ivermectin and polymer comprise crystalline ivermectin.

13. The method of claim 1, wherein the formulation further comprises a carrier comprising an oil and a gel and one or more pharmaceutically acceptable excipients.

14. The method of claim 13, wherein the gel comprises one or more of polymeric hydrocarbon gelling agents, poloxamer 407, carbomer, methylcellulose, and sodium carboxymethyl cellulose.

15. The method of claim 1, wherein the formulation further comprises a mineral oil.

16. The method of claim 1, wherein the formulation has a viscosity between about 30,000 cP and about 100,000 cP preferably between about 40,000 cP and about 90,000 cP.

17. A solid dispersion in the form of particles consisting essentially of ivermectin and a polymer to protect the ivermectin in the particles from sterilization and control the release of the ivermectin from the particles, wherein the ivermectin is in an amorphous form, the particles have a D90 particle size below about 10 microns preferably between about 800 nm and about 4 microns, and the ratio of ivermectin to polymer in the particle is about 1:10 to about 10:1 preferably from about 1:3 to about 4:1.

18. The solid dispersion of claim 17, wherein the polymer comprises PVP VA-64 and the PVP VA-64 is present at a ratio of ivermectin to PVP VA-64 of about 1:1.

19. The solid dispersion of claim 17, wherein the polymer comprises PVP K-30 and the PVP K-30 is present at a ratio of ivermectin to PVP K-30 of about 1:3.

20. The solid dispersion of claim 17, wherein the polymer comprises HPMC-E4M and the HPMC-E4M is present at a ratio of ivermectin to HPMC-E4M of about 4:1.

21. The solid dispersion of claim 17, wherein the particles comprise a first population of particles comprising a first ratio of ivermectin to polymer in the particle and a second population of particles comprising a second ratio of ivermectin to polymer in the particle and the first ratio and the second ratio are different, whereby the first population of particles releases the ivermectin faster than the second population of particles.

22. The solid dispersion of claim 21, wherein the D90 of the first population of particles is different from the D90 of the second population of particles.

23. The solid dispersion of claim 17, wherein the particles comprises a first population of particles comprising a first polymer in the particle and a second population of particles comprising a second polymer in the particle and the first polymer and the second polymer are different, whereby the first population of particles releases the ivermectin faster than the second population of particles.

24. The solid dispersion of claim 23, wherein the D90 of the first population of particles is different from the D90 of the second population of particles.

25. A pharmaceutical formulation in the form of a gel comprising the solid dispersion of claim 17 and a carrier in which the solid dispersion is insoluble or of minimal solubility, wherein the formulation has a viscosity between about 30,000 cP and about 100,000 cP preferably between about 40,000 cP and about 90,000 cP.

26. The pharmaceutical formulation of claim 25, wherein the carrier comprises one or more of poloxamer 407, carbomer, methylcellulose, sodium carboxymethyl cellulose and mineral oil with hydrocarbon gelling agents.

27. The pharmaceutical formulation of claim 26, wherein the hydrocarbon gelling agents comprise Ethylene/Propylene/Styrene Copolymer and Butylene/Ethylene/Styrene Copolymer.

28. The pharmaceutical formulation of claim 25, wherein the formulation releases the ivermectin over a period up to of twelve hours according to standard dissolution testing methods.

29. A method of killing demodex mites by topically applying the pharmaceutical formulation of claim 25 to the cutaneous tissue surrounding the eyelash, eyelid and/or to the eyelash or eyelid.

30. The method of claim 29, wherein applying the pharmaceutical formulation further comprises avoiding contact with the conjunctiva or cornea.

31. A kit comprising the pharmaceutical formulation of claim 25 and a precision applicator.

32. The kit of claim 31 wherein the precision applicator is designed to apply the formulation to the cutaneous tissue surrounding the eyelash, eyelid and/or to the eyelash or eyelid.
